(19) United States
(12) Patent Application Publication
(10) Pub. No.: US 2024/0091421 A1
(43) Pub. Date: Mar. 21, 2024

(54) SMART LACT8

(71) Applicant: San Jose State University Research Foundation, San Jose, CA (US)

(72) Inventors: Lin Jiang, San Jose, CA (US); Yuying Li, San Jose, CA (US); Ish Gulati, Cupertino, CA (US)

(21) Appl. No.: 18/469,330

(22) Filed: Sep. 18, 2023

Related U.S. Application Data
(60) Provisional application No. 63/376,245, filed on Sep. 19, 2022.

(51) Int. Cl. A61M 1/06

(52) U.S. Cl. CPC A61M 1/06935; A61M 1/064; A61M 1/0697; A61M 2205/3331; A61M 2205/3368; A61M 2205/36; A61M 2205/581; A61M 2205/583

(57) ABSTRACT

A breast pump and method of manufacturing the breast pump are described. The breast pump includes a pair of pads configured to be applied to human breasts of a user, each of the pads containing sensors and actuators, a pressure generator configured to provide vacuum within a first predetermined range and compression within a second predetermined range to operate the actuators in each pad, a temperature control system, and an artificial intelligence (AI) control system configured to control, based on feedback from the sensors, the pressure generator to mimic intra-oral motion of an infant and the temperature control system.

### PRIORITY CLAIM

This application claims the benefit of priority to U.S. Provisional Patent Application Ser. No. 63/376,245, filed Sep. 19, 2022, which is incorporated herein by reference in its entirety.

### TECHNICAL FIELD

Embodiments pertain to breast pumps. In particular, some embodiments relate to intelligent breast pumps.

### BACKGROUND

About 140 million are born each year, about 4 million of which are born in the United States. According to the most recent data from the Centers for Disease Control and Prevention (CDC), about 84% of the mothers in the United States initially choose to supply human milk to their infants due to a variety of reasons. The reasons include, for the infants, that such milk provides the best nutrition and immune support. For new mothers, on the other hand, prolonged and successful lactation has been shown to reduce postpartum depression in addition to increased bonding with their child.

One of the benefits that spurred the development of the breast pump was to make breastfeeding more convenient for mothers. Despite this laudable goal, however, many women experience adverse and dangerously discomforting side effects with currently available breast pumps.

Accordingly, it would be desirable to provide a well-designed, customized, and personalized breast pump to support breastfeeding mothers with a safe, comfortable, and portable milk expression process compliant with best hospital or return-to-work practices.

### BRIEF DESCRIPTION OF THE FIGURES

In the figures, which are not necessarily drawn to scale, like numerals may describe similar components in different views. Like numerals having different letter suffixes may represent different instances of similar components. The figures illustrate generally, by way of example, but not by way of limitation, various embodiments discussed in the present document.

FIG. 1 illustrates a smart breast pump system in accordance with some embodiments.

FIG. 2 illustrates another smart breast pump system in accordance with some embodiments.

FIG. 3A illustrates a schematic view of a pad in accordance with some embodiments.

FIG. 3B illustrates the pad design of FIG. 3A along the A-A line shown in FIG. 3A in accordance with some embodiments.

FIG. 4 displays another SPA pad design in accordance with some embodiments.

FIG. 5A displays master molds in accordance with some embodiments.

FIG. 5B displays a soft pneumatic actuator (SPA) pad mold in accordance with some embodiments.

FIG. 6 illustrates a method of fabricating an SPA pad in accordance with some embodiments.

FIG. 7 illustrates a cross-sectional view of a sensor structure in accordance with some embodiments.

FIG. 8 illustrates a method of fabricating a piezoelectric sensor in accordance with some embodiments.

FIG. 9A illustrates a front view of a sensor mold in accordance with some embodiments.

FIG. 9B illustrates a back view of a sensor mold in accordance with some embodiments.

FIG. 10 illustrates sensor structure fabrication in accordance with some embodiments.

FIG. 11A illustrates individual air channel design in accordance with some embodiments.

FIG. 11B illustrates a CAD model of the air channels implemented in a SPA pad in accordance with some embodiments.

FIG. 12A illustrates a front view of air channel structure in accordance with some embodiments.

FIG. 12B illustrates a back view of the air channel structure in accordance with some embodiments.

FIG. 13A illustrates pneumatic channels with sensors before bonding in accordance with some embodiments.

FIG. 13B illustrates pneumatic channels after bonding in accordance with some embodiments.

### DETAILED DESCRIPTION

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

A smart robotic breast pump system is described herein that provides self-adjusting compression and vacuum pressures as well as a temperature control system to perform a safe, efficient, and comfortable pumping experience for breastfeeding mothers. The breast pump system includes a plurality of components. The components include, but are not limited to: a) a pressure generator that combines peristaltic vacuum and positive pressure to replicate the mechanism of infant feeding on the mother's breast and thereby act more like a massage than continuous suction can provide; b) an artificial intelligence (AI) control system used to adjust the pump pressure in synchrony with milk production (based, e.g., on milk flow velocity) and comfort level of the user (identified, e.g., via user input); c) a temperature control system that provides comfort with a heating and cooling solution; and d) a personalized infant video and sound recording to aid with milk let-down reflex (personalized to the user such that video/sound of an infant of the user is provided). The breast pump may be portable or non-portable.

As above, one reason why mothers choose to supply human milk to their infants is because it provides unmatched nutrition and immune support for their infants. In spite of most mothers' intention to breastfeed, CDC1 statistics showed that 60% stop sooner due to hospital practices, work policies, and breast injuries. Early termination is largely associated with neurological and physical health issues for both mother and child. For example, infants with a cleft palate cannot create the pressure needed to suck milk from the nipple, preventing them from being breastfed, and infants with Down syndrome cannot control their oropharyngeal structure to latch on during breastfeeding. Breast engorgement and abscess, ductal blockage provide inadequate milk production and limited milk supply. Due to these reasons, a growing number of breastfeeding mothers use an electric or manual pump to support breast milk for their babies. Existing milk pumps usually extract the milk with only vacuum pressure, which is not similar to the infant's intra-oral movements. The actions of milk pumps and natural suckling have many differences; for example, the positive compression of the infant's palate, jaw, and tongue movement, which are important in breastfeeding.

Currently available breast pumps thus have a number of adverse side effects. One of these adverse effects, due to the use of suction-only (vacuum-only) mechanisms to extract milk, causes breast injuries including nipple soreness, breast tissue damage, and lactation complications after pumping. In fact, one survey based on 1,844 mothers indicated that about 62% reported pump-related problems and 15% reported injuries after breast pumping, at least in part due to commercially available breast pumps exerting much more pressure than that of an infant.

To overcome at least some of these issues, a bio-inspired breast pump prototype that can mimic infant suckling patterns, and thus provide maximal comfortable vacuum to produce the maximal milk yield, is described herein. The input vacuum pressure pattern and compression forces are inspired from term infants' natural oral suckling dynamics captured in prior clinical experiments. Open-loop input—output data are used to perform system identification for two different pumping stages that facilitates controller design for closed-loop stability and control. A physical breast pump with fuzzy-logic controlled soft pneumatic actuators and piezoelectric sensors may be calibrated and tested prior to use. Compression and vacuum pressure dynamics are coordinated to mimic the infant's feeding mechanism, which includes peripheral compression and oscillatory vacuum pressure. Experimental data on sucking frequency and pressure on a breast phantom shows consistency with clinical findings. All components described herein can be modified and customized based on the pumping frequency and the pressure feedback from the mother's breast, which assist in optimizing milk excretion with a comfort pumping experience.

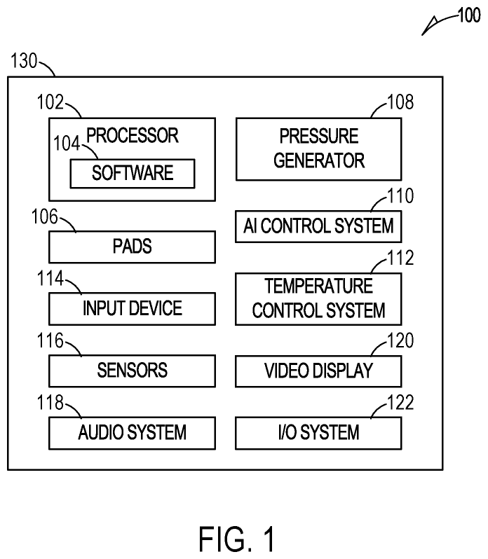

FIG. 1 illustrates a smart breast pump system in accordance with some embodiments. The smart breast pump system 100 includes a processor 102 on which software 104 is loaded to operate at least some of the components of the smart breast pump system 100. The components may include pads 106, a pressure generator 108, an AI control system 110, a temperature control system 112, an input device 114, sensors 116, and an input/output (I/O) system 122. In some embodiments, the smart breast pump system 100 may also include an audio system 118 and/or video display 120.

The components of the smart breast pump system 100 may be contained within a housing 130. The housing 130 may be a single integral structure or may be formed from disparate sections. Although the housing 130 is shown in FIG. 1 as including all of the components, in other embodiments, some of the components (e.g., one or more of the control, I/O, or audio systems or display) may not be contained within the housing 130.

In particular, the pressure generator 108 of the smart breast pump system 100 is configured to simulate an infant's oral suckling pattern using a SPA pad 106 to mimic an infant's oral compression on the breast, which is in line with a natural infant oral feeding mechanism, to provide an oscillatory pattern for positive oral pressures. The pressure generator 108 may provide a vacuum within a first predetermined range and a compression within a second predetermined range for use by the SPA pad 106. The first and second predetermined ranges may be the same or may be different (and may or may not overlap). For example, the first predetermined range (vacuum) and second predetermined range (compression) may be about 15-20 kPa and about 10-20 kPa, respectively. This is compared with a typical infant vacuum and compression of about 10-20 kPa and about 4-12 kPa, respectively. The particular pressure of each of the vacuum and compression may be selected by the AI control system 110 as discussed in more detail below.

The processor 102 may be used to control the intermated synchronize suction-compression-collection mechanism of the SPA pads 106 based on control signals from the AI control system 110. The use of SPA pads 106 is desirable due to the low cost, simple fabrication, and high customizable softness. The force, torque, motion range, and actuation speed of the SPA pads 106 may be measured to calibrate the system and provide inputs used to train the AI control system 110. The SPA pads 106, as described in more detail below, may contain air channels embedded in each pad 106 and connected with pneumatic tubes of the breast pump system 100. In particular, a vacuum pad with soft robotic contacts may result in the press and manipulation of objects upon pressurization. Air pulses with pressure control by the AI control system 110 may be used to mimic the infant intra-oral motion realistically. The vacuum and/or compression may be applied uniformly among the fingers or may be controlled individually to each finger using a separate valve in each connector part described herein.

Pad design considerations may include: elastomer wall thickness, air chamber size, contact area, shape, and size of the breast pump. In one embodiment, for a wall thickness of the SPA pad 106 of about 3 mm, a displacement is about 6 mm and the pressure range is expected to cover from about 30 kPa to about 40 kPa. Moreover, a force output range from about 0.5 N to about 2 N for about 6 mm displacement during the actuation.

The sensors 116 may be used to measure values for the AI control system 110 during training and non-training modes as feedback. The sensors 116 may include one or more pressure sensors to measure the vacuum and compression, one or more flow sensors to measure milk flow, and/or one or more temperature sensors, for example. In addition to the temperature feedback, vacuum feedback, and compression pressure feedback, the feedback may further include visual and/or audible feedback. The breast pump may generally operate in a typical manner, pumping milk from one or both breasts simultaneously and storing the milk in a container (e.g., bottle) attached to the breast pump through a conduit. An automatic alarm (visual, audible, and/or tactile) may be used to indicate the occurrence of an abnormal/predetermined event, for example the container has reached a predetermined amount of fill or the fill rate has fallen below a predetermined rate.

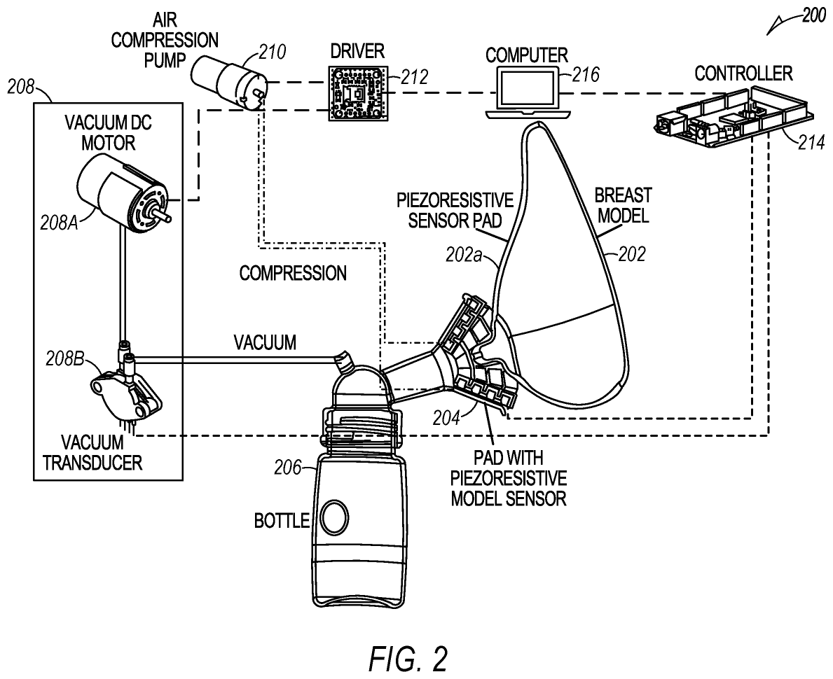

FIG. 2 illustrates another smart breast pump system in accordance with some embodiments. The smart breast pump system 200 may include components used for testing of the smart breast pump system 200. The smart breast pump system 200 may include a breast model 202 for simulation of an actual breast during normal operation.

To imitate the infant's oral behavior involving coordinated vacuum and compression pressure on the breast model 202, a finger-like soft robotic pad 204 is used. The robotic pad 204 includes pneumatic actuators, e.g., for each finger, and piezoelectric sensors were attached to the pneumatic actuators. One set of pneumatic actuators capture air pressure data in the chamber supplying vacuum to the robotic pad 204, while another set of piezoelectric sensors may be fabricated on a circular pad 202a and attached on the breast model 202 as a nipple shield to capture pressure data. The robotic pad 204 may be connected with a bottle 206 that retains liquid pumped from the breast model 202 (and may be identical to a bottle to be used in actual circumstances).

As shown, the smart breast pump system 200 may include a vacuum pump 208 that generates rhythmic intra-oral pressure supplied to the robotic pad 204. The vacuum pump 208 includes a vacuum motor 208a and a vacuum transducer 208b to convert the current from the vacuum motor 208a into pressure changes. The vacuum motor 208a may be, for example, a direct current (DC) motor as shown.

One or more pneumatic pumps 210 may be used to generate rhythmic compression pressure for the robotic pad 204. The pneumatic pumps 210 may be an air compression pump and use one or more solenoid valves to adjust the compressive force applied to the robotic pad 204. An inline pressure sensor may be used to capture air flow rates.

The smart breast pump system 200 may also include a driver 212 to drive the vacuum motor 208a and the pneumatic pumps 210 based on signals from a computer 216. The computer 216 may receive signals from a controller 214 coupled to and receiving sensor signals from the robotic pad 204 and the vacuum transducer 208b.

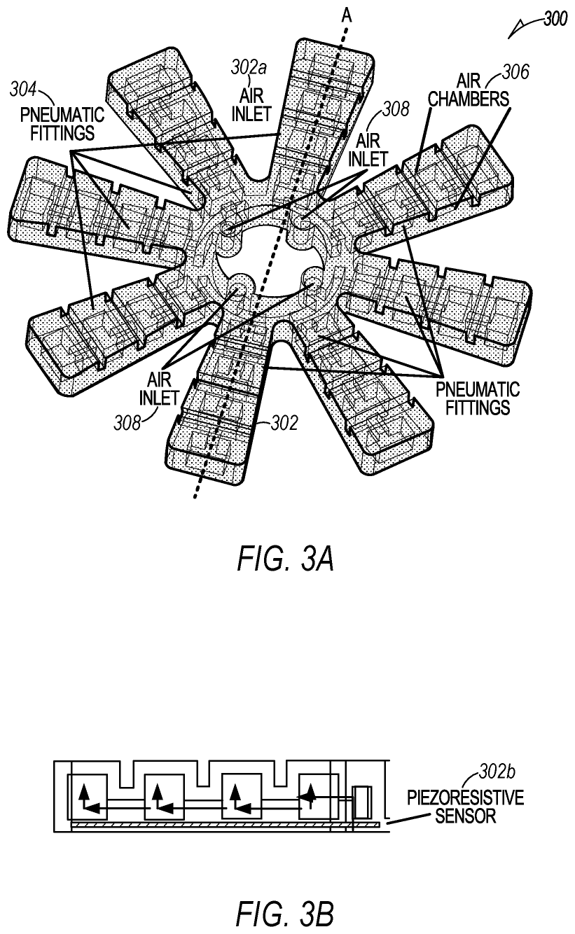

FIG. 3A illustrates a schematic view of a pad in accordance with some embodiments. FIG. 3B illustrates the pad design of FIG. 3A along the A-A line shown in FIG. 3A in accordance with some embodiments. The pad 300 includes multiple (e.g., 8) fingers 302 disposed regularly and symmetrically around an annular center for placement around a nipple. The annular center may be large enough to encircle an areola surrounding the nipple, for example about 100 mm. The shape of the pad 300 may emulate shapes used for breast massages for encouraging milk flow. The pad 300 may be formed from a medically approved material safe for human interaction such as platinum silicone, which is also semi-translucent. The material may be biocompatible, resistant to bacterial growth, and/or able to withstand high temperatures and pressures. In other embodiments, the fingers 302 may be asymmetrically disposed around the annular center.

Each finger 302 on the soft robotic pad 300 may be designed based on the elastomer wall thickness, air chamber size, contact area, shape, and size of the breast pump. Air inlets 302a to be connected to the vacuum pump may be symmetrically disposed around the annual center of the pad 300. Each finger 302 may have multiple air chambers 306 connected to at least one of the air inlets 302a. Four air chambers 306 in each finger 302 is shown in the embodiment of FIGS. 3A and 3B, although other numbers of air chambers may be used. In addition, each finger 302 may have one or more pneumatic fittings 304 coupled to at least one air chamber 306, whose movement is detected by a piezoresistive sensor 302b and supplied to a controller (not shown) for control of the compression and vacuum supplied to the pad 300. As shown in FIGS. 3A and 3B, a pneumatic fitting 304 is coupled to each air chamber 306. Although the number of air inlets 302a shown in FIG. 3A is smaller than the number of fingers 302, in other embodiments, the number of air inlets 302a may be equal to the number of fingers 302 in other embodiments.

The wall thickness of the pad 300 was about 3 mm and the displacement was about 6 mm in one embodiment. The pad may in some embodiments be fully actuate using about 30 kPa. In this embodiment, the force output ranged approximately from 0.5 to 2 N for 6 mm displacement during the actuation. Furthermore, the individual chamber area was calculated as 2N 30 kPa=0.000066 m2. The chamber width and height for each finger 302 in this embodiment was about 5 mm, and the air chamber length was about 7.5 mm.

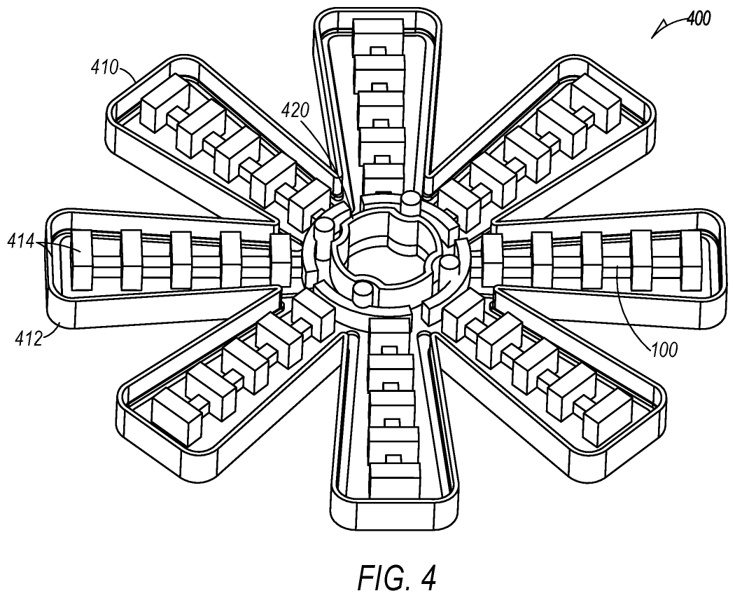

FIG. 4 displays a SPA pad design in accordance with some embodiments. As can be seen in FIG. 4, the flexible SPA pad 400 includes eight actuators 410 on a round plate 420. The plate 420 may have a diameter sized to provide support and actuation of the breast while leaving a sufficient amount of area for milk extraction. In some embodiments, the plate 420 may have a diameter of about 130 mm or 160 mm.

Other embodiments may use a different number of actuators 410 and/or the plate 420 may have a different shape. The different shape may be, for example, octangular, hexagonal, or rectangular in cases in which round actuators 410 overlap when fitting onto the cone shape shield (not shown).

The actuators 410 in some embodiments may be disposed symmetrically around the plate 420. Adjacent actuators 410 may be separated by the same angle (as shown about 45°). In other embodiments, at least some of the actuators 410 may be disposed non-symmetrically around the plate 420 (at least one of the actuators 410 is not centered between adjacent actuators 410). The actuators 410 may have an overall frustro-ovular shape as seen from a top view that extends radially from an outer surface of the plate 420. The actuators 410 may have a solid horizontal base 412 from which multiple vertical sections (or ribs) 414 extend. Although only 5 vertical sections 414 are shown, any number may be used. The SPA pad 400 provides pressure from the top and bottom of the breast. In some embodiments, an internal diameter of the elastomer pad may be about 20 mm to about 40 mm to fit on the nipple and areola area of a breastfeeding mother.

The SPA pad 400 also includes air inlets 416. In some embodiments, each air inlet 416 may be used by the pressure generator 108 to control a separate actuator 410. In other embodiments, each air inlet 416 may be used by the pressure generator 108 to control a pair of the actuators 410, for example a pair of the actuators 410 disposed symmetrically from each other across the plate 420 or adjacent actuators 410. The AI control system may actuate the actuators individually or in unison.

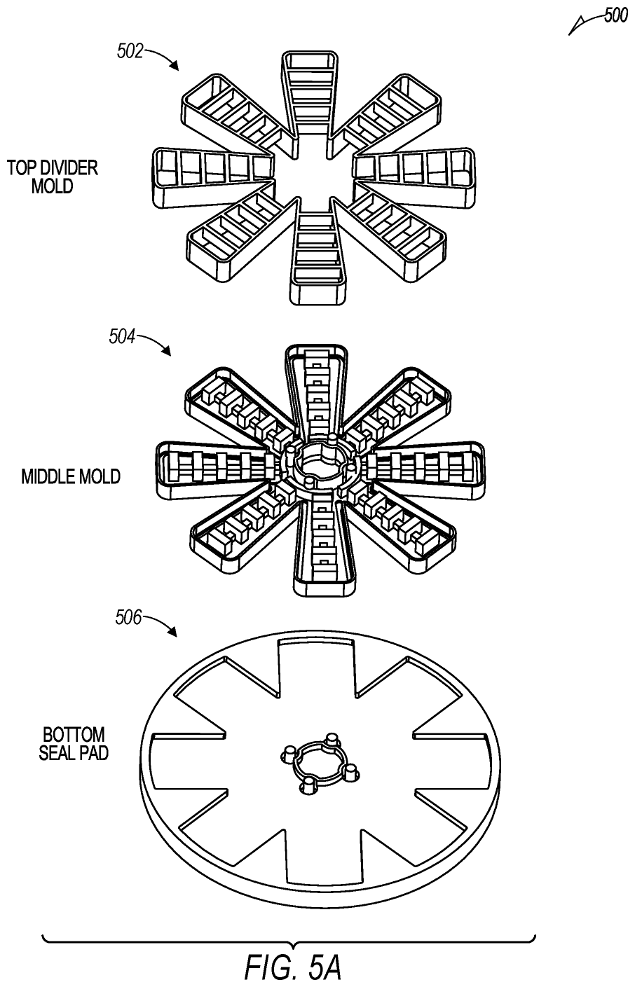

FIG. 5A displays master molds in accordance with some embodiments. The master mold structure 500 includes multiple individual portions to create the pad structure described herein. In particular, the master mold structure 500 includes a top divider mold 502, a middle mold 504, and a bottom seal pad 506. FIG. 5B displays a SPA pad mold in accordance with some embodiments. In particular, FIG. 5B shows the top divider mold 502, which includes eight actuators 510 on an octangular plate 520. To manufacture the SPA pad, SolidWorks™ model stereolithography/Standard Triangle Language or Standard Tessellation Language (STL) files were sent to PRUSA™ slicer software to generate G code files and then loaded to a PRUSA™ M3 3D printer to manufacture the molds. The molds were printed with Acrylonitrile Butadiene Styrene (ABS) material with a fine print setting. The molds were printed with PETG material with a detail print setting, with a layer height set to 0.10 mm.

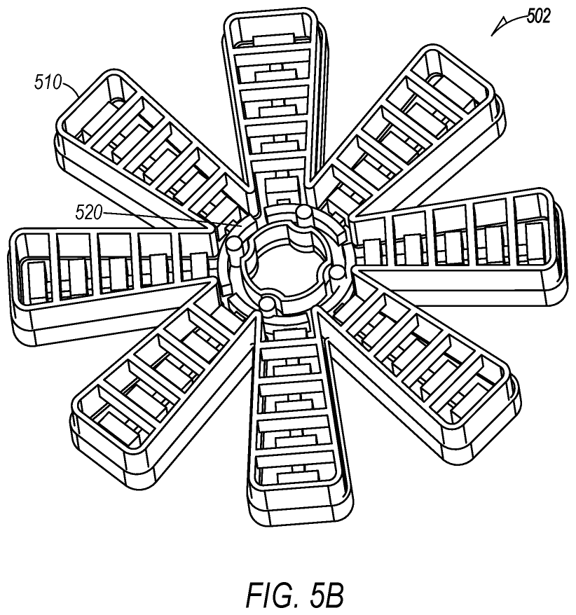

FIG. 6 illustrates a method of fabricating an SPA pad in accordance with some embodiments. The operations of the method 600 may be performed in the order shown or may be in a different order. To manufacture the actuator pads, the method 600 may employ a flexible material such as that above (e.g., Ecoflex™), a weight scale, a container, a vacuum pump, and a heating plate. In general, the method 600 may include layering the Ecoflex™ reinforcing the bottom seal, and embedding multiple (e.g., eight) piezoelectric sensors in the bottom seal. An initial operation is to form the SPA pad body part with the top and center mold. To form the SPA pad body part, at operation 602 the Ecoflex® may be measured and thoroughly mixed in a one-to-one ratio. The vacuum pump may be activated to de-gas the mixed solution at operation 604, which helps eliminate bubbles formed on the pad and avoid leak spots.

At operation 606, ease release may be applied (e.g., sprayed) onto the top divider mold 502 and the middle mold 504. In some embodiments, ease release may be applied prior to mixing and/or degassing of the solution. In some embodiments, ease release may be applied onto the bottom seal pad 506 at the same time.

The solution may be poured onto the top divider mold 502 and the middle mold 504 at operation 608 after the ease release is applied. Overflow may be prevented by filling gaps with a material such as clay.

The actuator chambers may then be formed at operation 612 using similar operations as that in operation 610 (an Ecoflex™ material may be measured, mixed, poured onto the molds, and de-gased). Similarly, the base mold may be formed at operation 614 using similar operations as that in operation 610 (an Ecoflex™ material may be measured, mixed, poured onto the base plate/bottom seal pad 506, and de-gased).

At operation 616 the soft robotic pad body and base are de-molded from the molds. The parts may be visually inspected for bubbles or rough surfaces.

After satisfactory inspection (if undertaken), the custom-made piezoelectric sensor may be inserted on and attached to the base pad at operation 618.

At operation 620, the actuator pad body (top elastomer part) and base elastomer piece may be bonded together. An additional layer of Ecoflex™ solution may, for example, as the bond. The mix, de-gas, and pour process of operation 610 may be repeated to seal the gaps between pieces. The top elastomer part may be lightly pressured down to ensure that the top elastomer part is in contact and sealed with the base elastomer part to form an assembly.

In some embodiments, the assembly may be placed on the hot plate to allow the mold to remain about 60° C. to speed up the curing process at operation 622. Whether or not the temperature of the assembly is increased by the application of heat, e.g., by the hot plate, the assembly may be cured.

After curing the assembly, the assembly may then be de-molded at operation 624.

Flexible Pressure Sensors Fabrication:

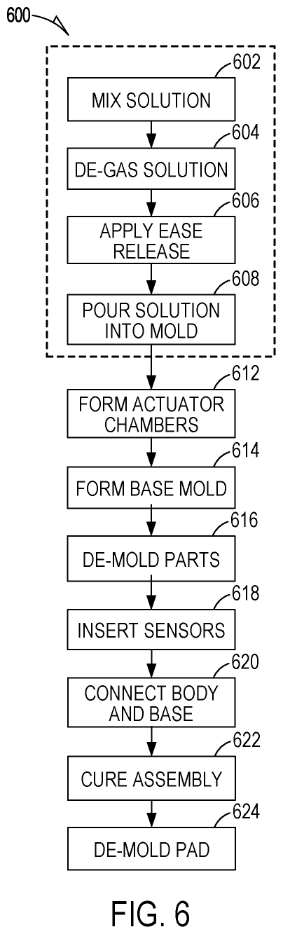

FIG. 7 illustrates a cross-sectional view of a sensor structure in accordance with some embodiments. The sensor structure 700 includes a flexible conductive fabric pressure sensor 722 was developed to bond with elastomer and embedded onto the soft pneumatic actuator or the elastomer pad. Each flexible conductive fabric pressure sensor 722 includes at least one piezo-resistive layer 710 that acts as a sensing layer and at least one actuating layer 720 to actuate the piezo-resistive layer 710. In other embodiments other number of layers may be used for one or more of these layers.

The piezo-resistive layer 710 may be formed from, for example, Velostat® and/or Adafruit®, and may be a conductive layer that changes its resistance value according to the amount of pressure acting on the piezo-resistive layer 710. The dimensions of the piezo-resistive layer 710 may be, for example, about 20 mm length by about 5 mm width by about 0.1 mm thickness for the pad dimensions indicated in the example above.

The actuating layer 720 may include at least one flexible conductive material 706, at least one conductive strip 708, and at least one wire 712 coupled to the flexible conductive material 706. The conductive strip 708 may be sandwiched between the flexible conductive material 706 and the piezo-resistive layer 710. The conductive strip 708 may be formed from, for example, a conductive fabric layer. The flexible conductive material 706 may be formed from copper tape and/or other conductive materials. As shown, the actuating layer 720 may sandwich the piezo-resistive layer 710.

The conductive strip 708 and flexible conductive material 706 may be used to improve the conductivity of the wire 712 acting on the piezo-resistive layer 710. The flexible conductive material 706 may be disposed to make electrical contact with the piezo-resistive layer 710 through the conductive strip 708 to carry current between the piezo-resistive layer 710 and the controller (not shown in FIG. 7).

The piezo-resistive layer 710 and actuating layers 720 sandwiching the piezo-resistive layer 710 may be sealed by and protected by a non-conductive adhesive 704 to form the flexible conductive fabric pressure sensor 722. One example of the non-conductive adhesive 704 may be single sided and may be transparent, for example clear tape. The thickness of the piezo-resistive sensor 722 may be about 0.3 mm in this example. A soft elastomer layer 702 may enclose the flexible conductive fabric pressure sensor 722 to form the sensor structure 700.

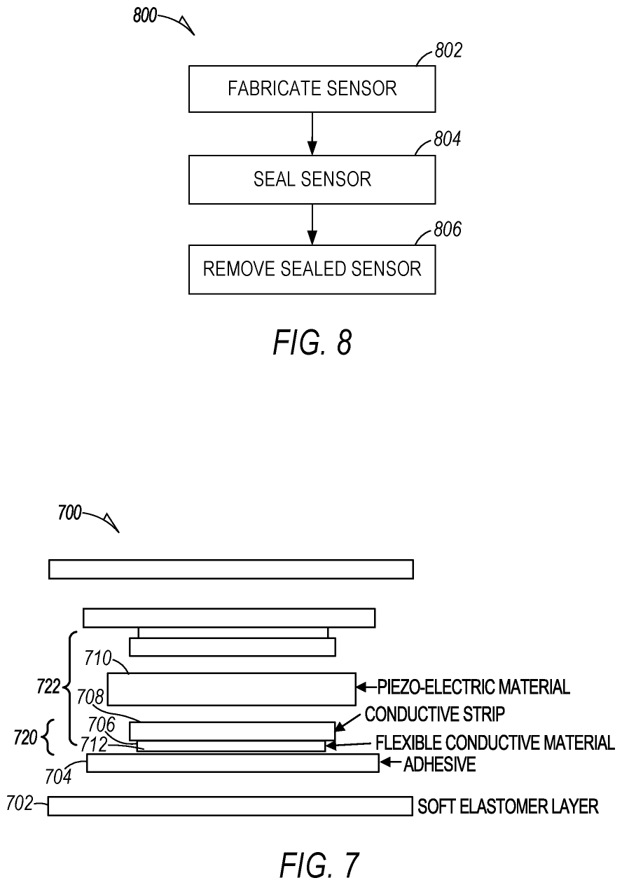

FIG. 8 illustrates a method of fabricating a piezoelectric sensor in accordance with some embodiments. The operations of the method 800 may be performed in the order shown or may be in a different order. To manufacture the piezoelectric sensor, at operation 802 the pressure sensor 722 is fabricated as above.

At operation 804, the pressure sensor may be sealed using soft elastomer layers. This may make bonding the structure to the soft robotic pads easier. To create flat elastomeric layers with flexible sensors, a 3D-printed mold with eight flexible pressure sensor slots on the front and a flat spindle cut-out on the back may be used. The mold may be formed in the pad shape (shown in FIG. 3A) and the flexible pressure sensor slots may be isolated from each other.

In one embodiment, a predetermined amount of elastomeric material may be deposited into the mold. The predetermined amount may be, for example, about 1 mL, and the elastomeric material may be, for example, Ecoflex® that has been de-gassed after mixing and prior to applying the elastomeric material to each slot. The mold may next be rotated so that the elastomer layer is evenly distributed within the mold using a spinner. For example, the spinner may be rotated at velocity (V)=about 500 rpm, acceleration (A)=about 50 rpm/s and time (T)=about 5 s. After spinning, the mold may be placed on a hot plate (set to about 60° C.) to speed up the curing process.

After curing the thin base elastomer layers (whether or not a hot plate is used), the pressure sensor may be disposed in each slot and another, thicker, layer of de-gassed elastomeric material disposed to cover the pressure sensor. The new elastomeric material layer may be, for example, about 3 mL and may be deposited with a syringe or other precision implement.

The mold may be placed inside a chamber to pull the vacuum pressure to about 1 bar. This may help force air between the pressure sensor and elastomer layer and allow the sensor to lay flat and centered between two elastomer layers. The elastomer may be cured and placed on the hot plate to speed up the curing process or left at room temperature based on the suggested curing time.

At operation 806, the pressure sensors may be removed from mold. The sensors may be removed one at a time or simultaneously.

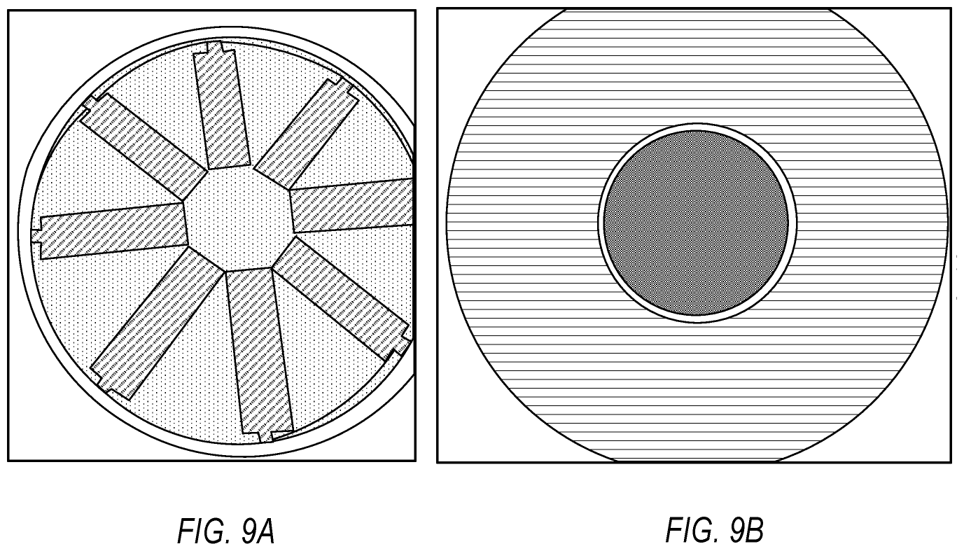

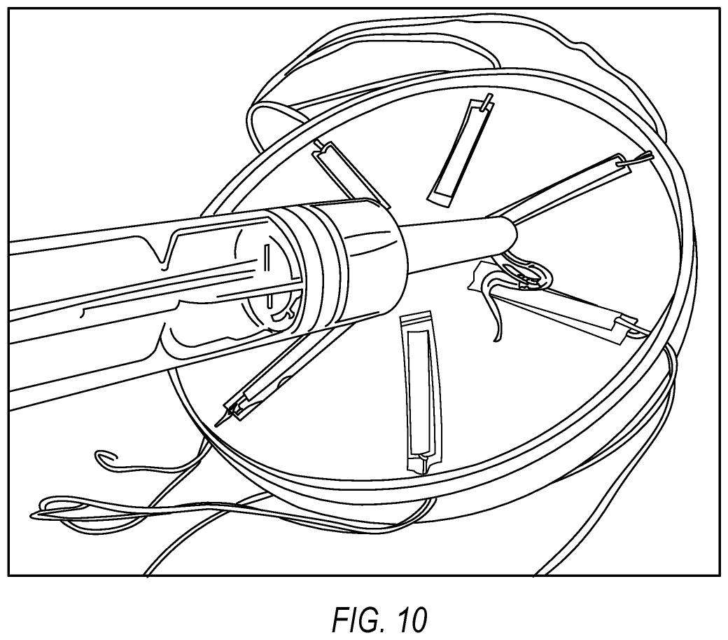

FIG. 9A illustrates a front view of a sensor mold in accordance with some embodiments. FIG. 9B illustrates a back view of a sensor mold in accordance with some embodiments. As shown in FIG. 9A, the mold includes eight flexible pressure sensor slots in the front and, as shown in FIG. 9B, a flat spindle cutout in the back. As above, during fabrication, eight conductive fabric sensors may be prepared, in one batch, and the pressure sensors sealed with clear tape. FIG. 10 illustrates sensor structure fabrication in accordance with some embodiments. FIG. 10 shows sealing of the sensors at operation 804 in FIG. 8. FIG. 10 shows sealing of the sensors at operation 804 in FIG. 8, in particular, deposition of the de-gassed elastomeric material to cover the pressure sensors.

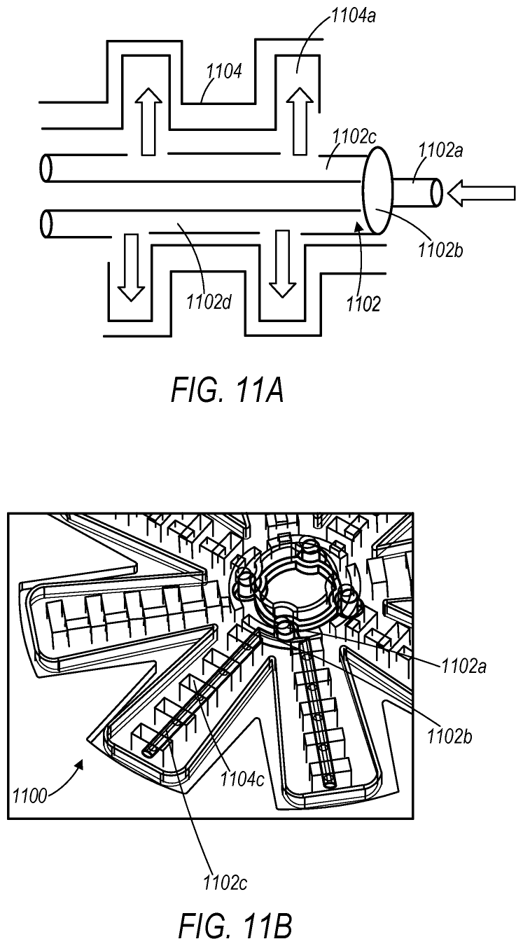

Pneumatic Connection part: A pneumatic connector part may be added with tubing to match the air chamber location to avoid leakage due to thin sections or blocked air channels. This allows delivery of sufficient air to the desired location on each soft pneumatic actuator. FIG. 11A illustrates individual air channel design in accordance with some embodiments. FIG. 11B illustrates a CAD model of the air channels implemented in a SPA pad in accordance with some embodiments. FIGS. 11A and 11B respectively provide additional cross-sectional and prospective views of the arrangement shown in FIGS. 3A and 3B within the SPA pad. As shown, the single air inlet 1102a of an air channel structure 1102 disposed at the center of the pad 1100 is separated at a connector part 1102b into two individual air channel tubes 1102c, 1102d each extending along an air channel of a different finger 1104 and configured to adjust the pressure in the air chambers 1104a in the finger 1104.

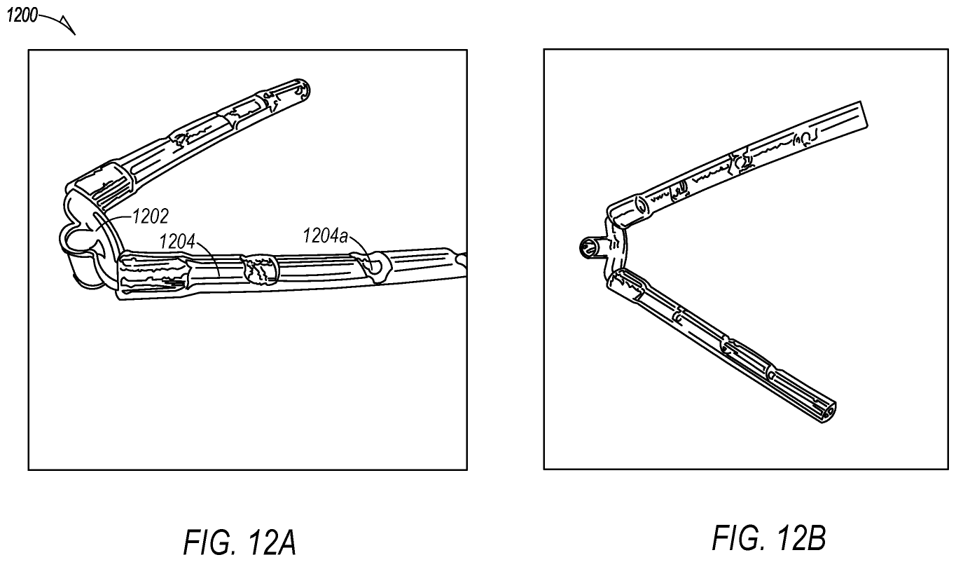

FIG. 12A illustrates a front view of an air channel structure in accordance with some embodiments. FIG. 12B illustrates a back view of the air channel structure in accordance with some embodiments. The air channel structure 1200 may include a connector part 1202 and a pair of air channel tubes 1204 that each contain a plurality of holes 1204a. The plurality of holes 1204a may align with the air chambers in FIGS. 11A and 11B. The air channel structure 1200 may be disposed in the pad as shown in FIG. 11B. The air channel tubes 1204 may extend at about 45°, depending on the number of fingers in the pad. The connector part 1202 may be formed by printing, for example. As the connector part 1202 may, in some embodiments, have a relatively thin wall thickness of about 0.5 mm, the Anycube™ 4X SLA printer is able to be used to 3D print the connector part 1202.

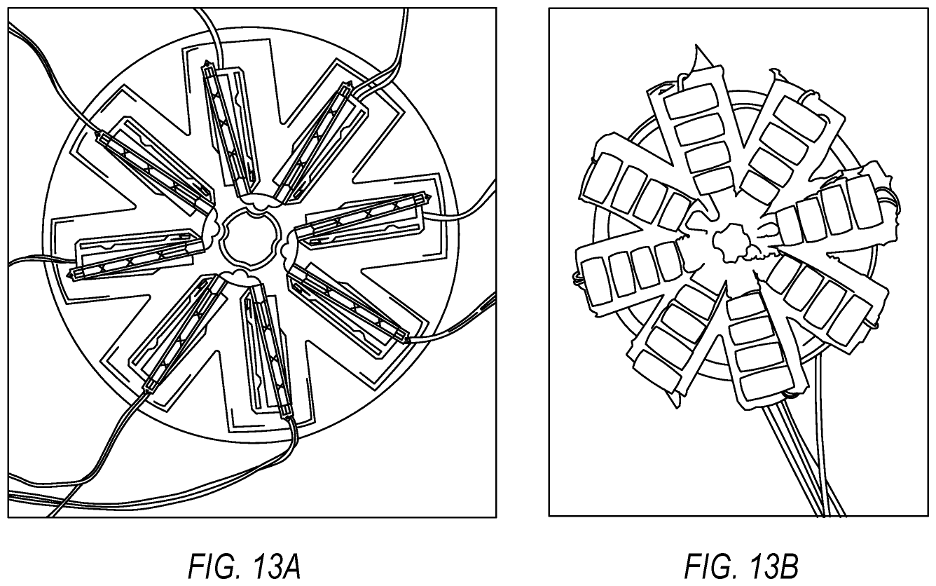

FIG. 13A illustrates pneumatic channels before bonding in accordance with some embodiments. FIG. 13B illustrates pneumatic channels after bonding in accordance with some embodiments. Pneumatic connection parts were implemented onto the SPA pad. The pneumatic tubing parts are implemented before connecting the top elastomer body to the base. After aligning the air channels on the elastomer body part, operations similar to the above are replicated in fabricating the SPA pad to bond the elastomer.

Turning back to FIG. 1, the AI control system 110 may be configured to use an AI algorithm to maximize milk production with a personalized comfort level. The AI control system 110 is configured to track outlet milk flow and control the pressure generator 108 to adjust both vacuum and compression pressure on the breast based on the milk flow rate and milk production. This may significantly reduce unnecessary vacuum extraction throughout the pumping period and relieve both the physical and mental stress on the mother when using a breast pump.

In particular, in some embodiments, the AI control system 110 may use data of a vacuum pressure pattern collected from the pressure sensors to match clinical data of the intra-oral vacuum pattern of an infant (adjusting the amplitude, duration, and frequency of the vacuum applied). Similarly, the AI control system 110 may use data from the temperature sensors to provide a temperature control accuracy above 80% for a temperature range of 25-37° C. The AI control system 110 may vary the vacuum strength and duration to provide, for example, a vacuum of about 190 millimeters of mercury amplitude with a 0.7-second duration and a one-impulse frequency per second.

In addition, the viscosity and density of human milk may decrease with increasing temperature. Thus, the use of a heating system in the smart breast pump system 100 may help for the milk removal process. For example, one study showed that a 10 mL of milk removal increment results in a one degree Celsius temperature increase on the nipple during the nutritive breastfeeding timeframe; during the breastfeeding process, mothers experience increases in body temperature with an average of 0.6-1.8° C., soreness, and breast pain during milk pumping. That is, temperature plays a substantial role during the milk removal process. Accordingly, a temperature control system 112 may be integrated into the breast pump system 100 and may be controlled by the AI control system 110. The temperature control system 112 may include a warming element for increasing effectiveness of milk removal and a cooling element for pain relief on the nipple after milk removal. Moreover, the cooling element may help slow the mammary blood flow to normal speed and to provide a comfortable post-pump experience to users. The heating element and/or cooling element may be disposed at individual separate or continuous areas within the smart breast pump system 100.

The AI control system 110 may be trained to provide optimal characteristics for a particular user. Specifically, as the optimal pulse amplitude, frequency, and duration may vary case-by-case for breast pump users, the AI control system 110 may include the pressure generator 108 to provide an adjustable vacuum power feature. Thus, the breast pump system 100 may include the temperature control system 112 to provide diverse comfort levels for different users as well as for the same user at different times. At least some of the sensors 116, such as temperature and strain sensors, may be integrated on the SPA pad 106. In some embodiments, a flexible force sensor array (FFSA) may be used in the SPA pad 106. The FFSA integrated system is capable of automatic detection and air pressure adjustment to match an ideal pressure. The temperature sensors may also be integrated into the SPA system alongside the strain sensor. A dual-mode pressure-temperature soft sensor may be used to simplify the feedback control system. Alternatively, one flexible sensor for sensing both pressure and temperature may be used to sense different temperatures (24.3-43.9° C.) and strains (0.1-50 kPa) based on the resistance variation.

An input device 114, such as a physical control input (e.g., buttons), an alphanumeric input device, or a voice-activated input device, may be provided on the smart breast pump system 100. In addition, visual and auditory stimulation may also be provided by the smart breast pump system 100, specifically via the audio system 118 and the video display 120. The stimulation may include infant sound and visual recordings personalized to the user of the smart breast pump system 100. Alternatively, or in addition, one or both of the audio system 118 and the video display 120 and/or the control inputs may be provided on a mobile device, via a personalized application (app) via the I/O system 122. The I/O system 122 may communicate with the mobile device using any communication protocol, such as 3GPP 4^{th} generation (4G), 5G, or later, WiFi, Zigbee, etc. The AI control system 110 may activate and deactivate the audio system 118 and/or the video display 120 based on or automatically upon activation and deactivation (respectively) of the breast pump system 100 or separately based on manual activation using the input device 114.

Soft pneumatic actuator pad testing: after determining that four pneumatic connectors are used on the SPA pad, two 6V peristaltic pumps may be selected to provide sufficient air to the actuator. In addition, two solenoid valves may be added to exhaust the actuators' air. The SPA pad may be connected to the electrical system. A separate power supply may be used to provide the 6V power for the testing.

Conductive heating fabric testing: high conductivity heater fabric EeonTex™ NW170-PI-20 were selected for the breast pump prototype's thermal system. The thermal diffusivity of the conductive heating fabric is 1.3×10² per square centimeter per second, thermal conductivity is 0.2 watts per meter per degree Kelvin, and specific heat capacity is 0.4 joules per gram per degree Kelvin. The time and length of the conductive fabric were the two parameters that varied in this test. Testing the temperature change for three different lengths of heating fabric in accordance with some embodiments shows that about a 19.5 mm length provides an optimal response among the tested lengths for the breast pump soft pneumatic actuators contact surface thermal system.

Flexible pressure sensor testing and calibration: since each inlet supplies air to two pneumatic actuator chambers, the two flexible pressure sensors within the chamber formed a half Wheatstone bridge to improve the circuit's sensitivity by a factor of two. The nominal resistance of each pneumatic actuator was measured initial test. When pressure was added to the sensor, the output voltage from eight flexible pressure sensors was translated into analog reading and output display on a serial monitor and serial plotter.

Sensor Calibration

Static and dynamic characteristics of the piezoelectric sensors may be calibrated. For sensor static characterization, increasing and decreasing calibration weight loads (0, 100, 200, 500, and 1000 g) may be applied to the force sensors with a sensor contact area of about 0.00042 m2, which provided a pressure range from about 0 to about 98 kPa. Accumulated data of static characterizations for both types of sensors indicated strong linearity among all sensors. A correlation function used for each sensor and converted into Arduino analogue readings, which were then used as inputs to the control system.

For dynamic characterization, 100, 200, and 500 g weights separately applied on the sensors and manually removed allowed testing of the sensor's ability to respond to pressure changes. A response time recorded when the pressure was instantly released showed that the time for the output to drop from 90% to 10% ranges from 0.03 to 0.07 s, less than the pressure profile updating rate at 0.1 s. These sensors proved to have sufficient dynamic characteristics for the measurement response purposes.

3.4. Complete Design, Control System and Experimental Setup

The complete experimental setup for testing the bio-inspired soft robotic breast pump with a control system included a flexible and human-tissue-mimicking breast phantom. A vacuum pump was pre-programmed to generated sinusoidal intra-oral pressure that follows the vacuum pressure profile of an infant based on clinical data. A pressure sensor was connected to the air loop to measure vacuum values in the tube. A simple closed-loop multiple-input multiple-output (MIMO) proportional-integral derivative (PID) control strategy controlled all motors with one central control system. Feedback from the vacuum transducer and custom-made piezoelectric pressure sensor pads were captured and imported to the analog reading pins on the microcontroller. Four piezoelectric sensors bonded on a silicone pad were used to measure the surface pressure from the nipple-areola area.

The real-time experimental control architecture includes a number of operations. The processor first identified the number and location of serial ports that were connected to the microcontroller. Vacuum and compression pressure profiles were pre-imported. A MATLAB program was employed to connect with the modules (e.g., Arduino™) and process and transmit real-time data to the hardware.

For the bio-inspired robotic breast pump, all experiments employed a user-defined profile to showcase the successful replication of infant suckling behavior. The vacuum frequency first started at 0.6 Hz, and then changed to 1.2 Hz. The local minimum and maximum vacuum for the first stage were −20 kPa and −8 kPa, respectively, whereas the local minimum and maximum vacuum for the second stage were −15 kPa and −5 kPa, respectively. Equations (1) and (2) demonstrate the pressure input profiles used.

$${P}_{vac .} = \{ \begin{matrix}0 & 0 \leq t < 85 s & \\ - 5 cos ( 2.4 π t ) - 10 & 85 \leq t < 190 s & Stage 1 \\ 0 & 190 \leq t < 230 s & \\ - 6 cos ( 1.2 π t ) - 14 & 230 \leq t < 500 s & Stage 2 \\ 0 & 500 \leq t < 520 s & \end{matrix} \qquad ( 1 )$$

$${P}_{comp .} = \{ \begin{matrix}0 & 0 \leq t < 85 s & \\ 5 cos ( 2.4 π t ) + 10 & 85 \leq t < 190 s & Stage 1 \\ 0 & 190 \leq t < 230 s & \\ 6 cos ( 1.2 π t ) + 14 & 230 \leq t < 500 s & Stage 2 \\ 0 & 500 \leq t < 520 s & \end{matrix} \qquad ( 2 )$$

Open-loop system identification experiment was conducted to extract the breast pump mechanical model using the sensor data. A two-staged experiment protocol was used to test the setup's feasibility. PID control parameters were selected based on the identified system and imported into the microcontroller unit. Tests were run utilizing the closed-loop control setup on the breast pump.

4.1. Soft Robotic Pad Actuation and Open-Loop System

Identification leakage checks were performed before each experiment. The deformation on the soft robotic pad was measured after being fully actuated by the air pump at 30 kPa. All eight pneumatic actuators were active. The chambers popped with pressure and provided compression to the mother's breast. When popped, most deformed air chambers were in the middle part of the fingers (the second and third chambers). The contact area of the pneumatic finger and the breast was about 10-15 mm by about 5-10 mm, comparable to the area of contact for an infant's tongue when latched on to their mother's breast.

The open-loop data acquisition was performed using various (Arduino™ and MATLAB™) code, the input voltage data in V and output pressure reading data in kPa were collected from the serial port communication between the processor and microcontroller. The collected data included noise and uncertainties. System identification was performed using offline open-loop system identification. All eight sensors worked and provided analogue data to the PC through a serial port communication to the microcontroller. The soft robotic pad on the breast pump is a non-linear structural material. Furthermore, there are various sinusoidal stages of pressure inputs. Hence, stochastic approximation algorithms were used to obtain the unknowns for the non-linear system at each stage. The inputs were the vacuum pressure and compression pressure. The outputs were the vacuum transducer data in the air loop and the averaged sensor data from all eight sensors on the soft robotic pad. Further PID controllers for each stage were designed based on the identified system. A recursive least squares (RLS) method was used for system identification. The estimated model was constructed as a second-order system presented in Equation (3),

y(k)=Σᵢ₌₁ⁿaᵢ⁰y(k−i)+Σᵢ₌₁ⁿbᵢ⁰u(k−i)+v(k) (3)
where y(k) is the output of the system at time k, u(k) is the input to the system at time k, a0i is the coefficient of the previous outputs in the system's response, b0i is the coefficient of the previous inputs in the system's response, and v(k) is the measurement noise at time k. Model coefficients for the system in two different stages are demonstrated in Table 1.

TABLE 1

| | Stage 1 | | Stage 2 | |
|---|---|---|---|---|
| System ID | Vacuum | Compression | Vacuum | Compression |
| [a₁⁰, a₂¹] | [−0.378, −0.493] | [−0.844, −0.084] | [−0.574, −0.271] | [−1.1615, 0.4471] |
| [b₁⁰, b₂¹] | [0.094, 0.103] | [0.134, −0.067] | [−0.109, 0.231] | [0.159, 0.096] |
| RMSE | 2.3721 | 12.363 | 3.5607 | 5.607 |
| Goodness | 91.77% | 68.8% | 74.88% | 74.45% |
| of Fit | | | | |

Data regarding system identification performance with RMSE and model fit are also presented in Table 1.

Stage 1 active pumping ran from time=85 s to time=190 s. Data for system identification was from time=100 s to time=130 s, and data for system validation was obtained from time=140 s to time=170 s. As shown in Table 2, the RMSE for stage 1 vacuum and compression was 2.3727 and 1.6509, respectively, indicating a goodness of fit during 100-130 s of pumping.

TABLE 2

| Parameters | Vacuum Control | Compression Control |
|---|---|---|
| Proportional, P | 0.011 | 167.3 |
| Integration, I | 0.109 | 334.6 |
| Derivative, D | 0.0157 | — |
| Settling time, s | 0.72 | 0.85 |
| Overshoot | 5.65% | 7.64% |
| Steady state error | 6.85% | 10.03% |
| RMSE for Stage 1, kPa | 2.3727 | 1.6509 |
| RMSE for Stage 2, kPa | 3.6544 | 3.1405 |

Stage 2 active pumping ran from time=230 s to time=500 s. Data for system identification was from time=250 s to time=280 s, and data for system validation was extracted from time=400 s to time=430 s. As shown in Table 2, the RMSE for stage 2 vacuum and compression was 3.6542 and 3.1405, respectively, indicating a goodness of fit during 140-170 s of pumping.

4.2. Closed-Loop Controller Design and System Performance

The mechatronic system for the SmartLac8 system enabled feedback controls for two air pump motors for soft robotic pad actuation and one vacuum pump for suction using the piezo-resistive sensor data from the soft robotic pad and the piezo-resistive sensor on the breast phantom. A closed-loop feedback control system running on a microcontroller provided the real-time robust coordination of the soft robotic system. The microcontroller interacted with the vacuum and compression pump motors through the MATLAB™ simulink using the tuned PID parameters following system identification. The control system was designed with two levels. The first level collected control feedback signals from the motor encoder, pressure sensors and vacuum pressure transducer. In the second level, real-time set points were tracked from the system input using the tuned PID parameters on the Arduino board.

Table 2 lists the PID parameters, response time and tracking error for the vacuum pump control and compression air pump control for dynamic-pressure-tracking performance generated by the oral breast suckling simulator under various pressure frequency. Within 0.7 s and 0.8 s after frequency variances for the vacuum and compression pressures, respectively, the tracking error decreased to ±10%. No significant magnitude changes were observed, and the frequency change corresponded to the input pressure dynamics. Although vibrations and environmental errors still affected the results of real-time control, both pumping systems were stable after onboard PID tuning. Hence, the breast pump achieved the desired frequency tracking in one suck cycle where the frequency ranged from 0.6 to 1.2 Hz in two stages. The average compression pressure on the breast was 12.25±5.42 kPa.

The designed architecture contains a soft robotic pad with eight finger pneumatic actuators controlled individually with air pumps, linear motors for vacuum and air pumping, custom-made piezo-resistive sensor pads, digital pressure transducers, and onboard feedback controllers with a proportional integration-derivative (PID) control algorithm. In particular, a MATLAB™ program linked with Arduino™ hardware was developed for real-time data monitoring and feedback control. Dry laboratory tests on the reliability and robustness of the breast pump confirmed the better performance with the feedback control loops for vacuum pressure tracking and non-linear soft robotic control. The bio-inspired breast pump, i.e., SmartLac8, successfully mimicked infant oral suckling behavior with a better fidelity.

The onboard PID control was based on the transfer function of the motors, generated by open-loop system identification. Advanced real-time PID control is more precise in tracking the vacuum and compression pressures but also increases the computational cost. The current maximum percentage error in the simulator was <10%, acceptable as a preliminary device for feasibility tests.

All electronics are actuated under 5 V, making it safe, portable, and convenient to use. Integrated with an advanced control unit, the breast pump is the first fully controllable device that replicates the biomechanics of breastfeeding and functions as an educational, training, and research tool. The compression forces may be tuned based on varying the vacuum frequency and strength to match with the physiological mechanics of an infant's oral suckling during breastfeeding and provide mothers both comfort and emotional support. While pumping, mothers can input and change their desired pressure for vacuum suction and compression pressure. Furthermore, it is notable that the design robustness of the breast pump allows for a personalized, easy-to-use, reproducible advance robotic pump for mothers, as it can provide a controllable and tolerable pressure range due to the effective vacuum and compression pressure division and coordination.

### Examples

Example 1 is a breast pump comprising: at least one pad configured to be applied to a human breast, the pad containing fingers; a pressure generator configured to provide vacuum within a first predetermined range and compression within a second predetermined range for application by the pad; a temperature control system in the pad; and a control system configured to control the pressure generator to mimic intra-oral motion of an infant and the temperature control system.

In Example 2, the subject matter of Example 1 includes, wherein the first predetermined range and the second predetermined range are different and overlap.

In Example 3, the subject matter of Examples 1-2 includes, wherein the pad comprises fingers extending radially from an annular center plate.

In Example 4, the subject matter of Example 3 includes, wherein each finger includes at least one actuator configured to move the finger and at least one sensor to detect movement of the finger.

In Example 5, the subject matter of Example 4 includes, wherein the at least one sensor comprises a piezoelectric sensor that includes a piezoelectric structure encased in an elastomer, the piezoelectric structure containing a piezoelectric material sandwiched between flexible conductive materials that are sealed by a non-conductive adhesive.

In Example 6, the subject matter of Examples 4-5 includes, wherein the at least one actuator comprises a plurality of separated air chambers actuated by compression and oscillatory vacuum pressure provided by the pressure generator.

In Example 7, the subject matter of Example 6 includes, wherein the air chambers are connected by an air channel through which the compression and oscillatory vacuum pressure is provided.

In Example 8, the subject matter of Example 7 includes, an air channel structure extending along the air channel, the air channel structure having holes disposed therein to align with the air chambers.

In Example 9, the subject matter of Example 8 includes, wherein the air channel structure is disposed between at least a portion of the at least one sensor and at least some of the air chambers.

In Example 10, the subject matter of Examples 8-9 includes, wherein the air channel structure comprises a pair of air channel tubes connected at a connector part disposed at the center plate, the air channel tubes extending along air channels of adjacent fingers of the pad, an air inlet of the connector part configured to provide the compression and oscillatory vacuum pressure to the air chambers of the adjacent fingers.

In Example 11, the subject matter of Examples 3-10 includes, wherein the fingers are arranged symmetrically around the center plate.

In Example 12, the subject matter of Examples 1-11 includes, at least one of an audio system or a video display configured to provide a recording of an infant.

In Example 13, the subject matter of Examples 1-12 includes, wherein the temperature control system comprises a warming element and a cooling element, and the control system is configured to control the temperature control system to provide warmth, via the warming element, to each pad to increase effectiveness of milk removal and cooling, via the cooling element, for pain relief on a nipple after milk removal.

In Example 14, the subject matter of Examples 1-13 includes, wherein the control system is trained to configured to control the pressure generator to provide, via actuators in the pads, a pulse amplitude, frequency, and duration optimized to a user of the breast pump.

In Example 15, the subject matter of Examples 1-14 includes, wherein at least one of the fingers includes at least one sensor to detect movement of the finger, the at least one sensor comprises a flexible force sensor array (FFSA) and temperature sensors configured to provide feedback to the control system for control of the pressure generator and the temperature control system, respectively.

Example 16 is a method of forming a breast pump, the method comprising: inserting sensors into a soft pneumatic actuator (SPA) pad to be applied to a human breast; placing a pressure generator in a housing to provide vacuum within a first predetermined range and compression within a second predetermined range for application by the pad; placing a temperature control system in the housing; and training an artificial intelligence (AI) control system to control the pressure generator to mimic intra-oral motion of an infant and the temperature control system.

In Example 17, the subject matter of Example 16 includes, D printer.

In Example 18, the subject matter of Example 17 includes, mixing a solution to be cured; degassing the solution using a vacuum pump; pouring the degassed solution into a mold; degassing the solution in the mold; curing the degassed solution to form a body part; placing the sensors on the body part; placing the body part with the sensors onto a base part; sealing the body part with the base part using another solution to form an assembly; and curing the assembly.

Example 19 is a breast pump comprising: a pad configured to be applied to a human breast, the pad containing sensors and actuators; a pressure generator configured to provide vacuum within a first predetermined range and compression within a second predetermined range to operate the actuators in the pad; a temperature control system in the pad that includes, a warming element and a cooling element; and an artificial intelligence (AI) control system configured to control, based on feedback from the sensors: the pressure generator to adjust a pulse amplitude, frequency, and duration of at least one of vacuum or compression to mimic intra-oral motion of an infant, and the warming element to increase effectiveness of milk removal and the cooling element to provide pain relief on a nipple after milk removal.

In Example 20, the subject matter of Example 19 includes, wherein: the pad comprises fingers extending radially from an annular center plate, each finger comprises a plurality of separated air chambers actuated by compression and oscillatory vacuum pressure provided by the pressure generator, and the air chambers are connected by an air channel through which the compression and oscillatory vacuum pressure is provided through an air channel tube of an air channel structure extending along the air channel.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Although an embodiment has been described with reference to specific example embodiments, it will be evident that various modifications and changes may be made to these embodiments without departing from the broader scope of the present disclosure. Accordingly, the specification and drawings are to be regarded in an illustrative rather than a restrictive sense. The accompanying drawings that form a part hereof show, by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

The subject matter may be referred to herein, individually and/or collectively, by the term “embodiment” merely for convenience and without intending to voluntarily limit the scope of this application to any single inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, UE, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A breast pump comprising:
at least one pad configured to be applied to a human breast, the pad containing fingers;
a pressure generator configured to provide vacuum within a first predetermined range and compression within a second predetermined range for application by the pad;
a temperature control system in the pad; and
a control system configured to control the pressure generator to mimic intra-oral motion of an infant and the temperature control system.

2. The breast pump of claim 1, wherein the first predetermined range and the second predetermined range are different and overlap.

3. The breast pump of claim 1, wherein the pad comprises fingers extending radially from an annular center plate.

4. The breast pump of claim 3, wherein each finger includes at least one actuator configured to move the finger and at least one sensor to detect movement of the finger.

5. The breast pump of claim 4, wherein the at least one sensor comprises a piezoelectric sensor that includes a piezoelectric structure encased in an elastomer, the piezoelectric structure containing a piezoelectric material sandwiched between flexible conductive materials that are sealed by a non-conductive adhesive.

6. The breast pump of claim 4, wherein the at least one actuator comprises a plurality of separated air chambers actuated by compression and oscillatory vacuum pressure provided by the pressure generator.

7. The breast pump of claim 6, wherein the air chambers are connected by an air channel through which the compression and oscillatory vacuum pressure is provided.

8. The breast pump of claim 7, further comprising an air channel structure extending along the air channel, the air channel structure having holes disposed therein to align with the air chambers.

9. The breast pump of claim 8, wherein the air channel structure is disposed between at least a portion of the at least one sensor and at least some of the air chambers.

10. The breast pump of claim 8, wherein the air channel structure comprises a pair of air channel tubes connected at a connector part disposed at the center plate, the air channel tubes extending along air channels of adjacent fingers of the pad, an air inlet of the connector part configured to provide the compression and oscillatory vacuum pressure to the air chambers of the adjacent fingers.

11. The breast pump of claim 3, wherein the fingers are arranged symmetrically around the center plate.

12. The breast pump of claim 1, further comprising at least one of an audio system or a video display configured to provide a recording of an infant.

13. The breast pump of claim 1, wherein the temperature control system comprises a warming element and a cooling element, and the control system is configured to control the temperature control system to provide warmth, via the warming element, to each pad to increase effectiveness of milk removal and cooling, via the cooling element, for pain relief on a nipple after milk removal.

14. The breast pump of claim 1, wherein the control system is trained to configured to control the pressure generator to provide, via actuators in the pads, a pulse amplitude, frequency, and duration optimized to a user of the breast pump.

15. The breast pump of claim 1, wherein at least one of the fingers includes at least one sensor to detect movement of the finger, the at least one sensor comprises a flexible force sensor array (FFSA) and temperature sensors configured to provide feedback to the control system for control of the pressure generator and the temperature control system, respectively.

16. A method of forming a breast pump, the method comprising:
inserting sensors into a soft pneumatic actuator (SPA) pad to be applied to a human breast;
placing a pressure generator in a housing to provide vacuum within a first predetermined range and compression within a second predetermined range for application by the pad;
placing a temperature control system in the housing; and
training an artificial intelligence (AI) control system to control the pressure generator to mimic intra-oral motion of an infant and the temperature control system.

17. The method of claim 16, further comprising printing molds of the SPA pad from Acrylonitrile Butadiene Styrene (ABS) material using a 3D printer.

18. The method of claim 17, further comprising:
mixing a solution to be cured;
degassing the solution using a vacuum pump;
pouring the degassed solution into a mold;
degassing the solution in the mold;
curing the degassed solution to form a body part;
placing the sensors on the body part;
placing the body part with the sensors onto a base part;
sealing the body part with the base part using another solution to form an assembly; and
curing the assembly.

19. A breast pump comprising:
a pad configured to be applied to a human breast, the pad containing sensors and actuators;
a pressure generator configured to provide vacuum within a first predetermined range and compression within a second predetermined range to operate the actuators in the pad;
a temperature control system in the pad that includes a warming element and a cooling element; and
an artificial intelligence (AI) control system configured to control, based on feedback from the sensors:
the pressure generator to adjust a pulse amplitude, frequency, and duration of at least one of vacuum or compression to mimic intra-oral motion of an infant, and
the warming element to increase effectiveness of milk removal and the cooling element to provide pain relief on a nipple after milk removal.

20. The breast pump of claim 19, wherein:
the pad comprises fingers extending radially from an annular center plate,
each finger comprises a plurality of separated air chambers actuated by compression and oscillatory vacuum pressure provided by the pressure generator, and
the air chambers are connected by an air channel through which the compression and oscillatory vacuum pressure is provided through an air channel tube of an air channel structure extending along the air channel.
